SEQUENCE LISTING (17 sequences)

SEQ ID NO: 1 - DNA, 28 bp, Artificial Sequence
  Primer 1 of FBP7 promoter
cgaagcttcatcaaactaaattaatact

SEQ ID NO: 2 - DNA, 28 bp, Artificial Sequence
  Primer 2 of FBP7 promoter
cgtcgacttcaaagcctttcagaaacct

SEQ ID NO: 3 - DNA, 495 bp, Petunia
catcaaactaaattaatactctggaatattagcatgcgtgaatgtcgtcgtttgtcaattgaacttcactaatagtgttaaacaatagatacagtgataaagaatagatacagtgatacaaaatcaggtcatcctgctaatcacactacactaatagtgtaaataattaaactaaagatttaaatagaagagggggcatattagtcaagttgaaagcaagtaacacgctttgcaatttttggtgaaattctggtctgcagtaaagcagcgtacaaggccacgtctggtttatatctggaaaaaggccatttcaatgtcattttcttggccagctctttctttcctttaattttaagtgatagttaaagctagggtaggtatttataaggaaagaaagacattttaagtattgagtttttcctcattcattagtgttgtaattccaagactacaaagtctttggaagtacgtgtgaggtttctgaaaggctttgaa

SEQ ID NO: 4 - DNA, 27 bp, Artificial Sequence
  Primer 1 of E6 promoter
cgaagcttaattcataggtataggaag

SEQ ID NO: 5 - DNA, 25 bp, Artificial Sequence
  Primer 2 of E6 promoter
cgtcgacggctatggttgctaatgc

SEQ ID NO: 6 - DNA, 1454 bp, Gossypium hirsutum
aattcataggtataggaagaagccctgtcaaatagagattttttctttcgaccatatttcgattgttaatacgatatataaggaccgctactacaaatagtactacacccttgatcggaaatatcgattgcttgttgaaccctgtgaattgcgtgaaagtaggatactccaaattcgggggtccaagagttttataaaacgttcttggtggaaaaaaatgtgaataaagatcccactgaattgaattgggtccatgaatctaagaaatagtgagaattctcctatttttatttatttatttgcttaggaagttttactgacactgcttttatttttccatcaatcaaatttaagagacaattcactttttataattaacaaaaaaaaacaaaaagaaaataaaagaaattacttttttctttttcgtgttcgatacaagatagatgaaatatcaaaaataaaatgaaatgaaaatatattactagtgatatatcacctccattatgtaggggaaagaaataaaaataatattaatttatgatacttccataatgtggttaaaaataattatctagtatttttttgtaaaaaaaaaaagttgatatctatgctactaatgaggtttcttagtgagtttgttactactaataaagtttatttgcatggttgagaccttatgcttttcaaatacccatttttgaattttaaaaattgtgaatttttattatatttaaaaaacaagttatttatataactagtaatgtattattttgacttttttttaatcgagttaatgttggttatttcgttataccaattcaataaaatattttatttatataaattatagcatactcacgatgtgggtgaagtaaaattatttaacaaatatattttgaaaaattgataaaaatactaaatgaggttttggttgaatagtaagatataattattacaaattataaatatgtaggttcaaaatctatcatgtgtatatttgtactattattctatataaattgataaccttataaaagtatctaatttagtttatggttgattgatcgataataccaaatttattaaaaattaatattagtaaagatatatagtacaaaactaaacataaaattttatatgttaaggaaatagcggaaaaaatatcatatttgtagaactgtttagcagtgtgggagaatgggatcattacaagcaaaaatgaaatatatatcattaataacaaacataaaagaaagcgtcttttgataaagttgttattggtgtaatgtgaagggaccacaatcatcaccattcaccacttgctcctaattgagttgaaatctttttacaacatagaaaactagaagatcgccctttcttgcttcatatatatagattttgtatcatcgcaatttcacatcacacacacaagtaaagcattagcaaccatagcc

SEQ ID NO: 7 - DNA, 27 bp, Artificial Sequence
  Primer 1 of Agl5 promoter
cgaagcttctcagtaccacttaactgc

SEQ ID NO: 8 - DNA, 25 bp, Artificial Sequence
  Primer 2 of Agl5 promoter
cgtcgacctacccaactagagcatc

SEQ ID NO: 9 - DNA, 2065 bp, Arabidopsis
ctcagtaccacttaactgctgcttcaactttattttttgtttttggtttctgttttggtttataatttcttttagaggaatatttctttggaagtatttaaaaacccatttgcagaaaacagaaggtttggtagttttttttcaaactgaagtgttatagaaggaaaaagaaagaaagaacatggaccattacttctaacattttctcaaatatcacacacctaattcttttaattaaattctatatgcatacatttagattgaaatttgaaattgattggaattaagtggtagaaaatatcaaattagaaagaagaaaaaagaaagaaagaagacagttgggtttggattgggtggggagagagcattgaaggaggaccatccaatttttctttcatgtttagaggaaaaaagagaggtatgggctctgtttctcatcagaatcaccttttggaaactttccttttttggtaatccctttttattctccttttctttttattgccctactcctatatacattgcatgtcgttaatatttacctggcattttatatataaaaaaaatatttccaattaaaccaatatttagctaatatgctaagaatttgggtggtttatatgagtgttcgtcgtcaacgttagaatggggttatatgaatatgagtgttataacttgtcaactaccaagcgagacaatgaataagatttcagtgttcttgccaattaaaaaaactgtaaatagtatatattattatttggttgtgttcagaaatggcaatttaagagtatatattataactgggttagtttttccaaaattcttatgttcaagtcttaatcttggacattaggttagtccaacggcaatttgaatatttattttgataattaattacatcatattacgtaatttagacaaaagaactacatcatatatgcgacaatataattagagaagatgaaactaataaagtaaatgaaagcagagagcccacggggaatgactaatcatgtgtgtagtcattatccatgaaccctttctcagatttgcattaattaattgtagttacgaaaggaaactcttttgtgttttgcccattcctcatcattttcattgtacttctattcaattttcttaacgagcaacacacattttcacattcatgtcgaattaattaagttggaaattctgcaaaatcatttcaagaaagaacttgatgagagctggtaggtcttttgattgactttcgtggtctatattctcaatatatagtatatgctttgaaagttgaatatgcactagttcttgttgtgttaagagttaattagcgtgaagtaatataattctcatatcgagtgtttaatttactagctttatgacaaaataatcataatcaaatagaatactaatgaagaaaaaggtaatatatatatgtaagaaagatgaattatgtaaagattgcttttactagccctgtgacaaaaaatgttaaatctttatcaaattgaacgatgattgataattgaaatttaattttaatgaaaagaagtagtatataaatgggtaagaaagagagatttccaaattaaaggacaggtgatttttactcaagtatcataatcaaattgtgtatacgtccaatctcattaattagtgcataagttattgatcactaactcttgtgaaaaagaaaagaatcttttgttttgacgaaaacatattccaaataccattcatagggatactaaacccaagactttggatttcccttcatcagaaattttccaacgtttactttgagatatttttttcttcttagaacatataataaataggttcatgaaaacgtaacaatgcgttggagattctttacctggcttgttgccttaactcccacaaagattcttttgcttgaagaaatttcaagcttttgtgtatatatccaacgtcacagtgctaaagacgtttgagaaagtttctgttgaaagaagagtaaaaactcaaaaagaaaatgggtttcattattgcgattgcaaaacgttttgatgctctagttgggtag

SEQ ID NO: 10 - DNA, 27 bp, Artificial Sequence
  Primer 1 of iaaM gene-KpnI
ggtaccatgtcaccttcacctctcctt

SEQ ID NO: 11 - DNA, 26 bp, Artificial Sequence
  Primer 2 of iaaM gene-EcoRI
gaattctaatttctagtgcggtagtt

SEQ ID NO: 12 - DNA, 2279 bp, Agrobacterium tumefaciens
ggtaccatgtcaccttcacctctccttgataaccagtgcgatcatctcccaaccaaaatggtggatctgacaatggtcgataaggcggatgaattggaccgcagggtttccgatgccttcttagaacgagaagcttctaggggaaggaggattactcaaatctccaccgagtgcagcgctgggttagcttgcaaaaggctggccgatggtcgcttccccgagatctcagctggtggaaaggtagcagttctctccgcttatatctatattggcaaagaaattctggggcggatacttgaatcgaaaccttgggcgcgggcaacagtgagtggtctcgttgccatcgacttggcaccattttgcatggatttctccgaagcacaactaatccaagccctgtttttgctgagcggtaaaagatgtgcaccgattgatcttagtcatttcgtggccatttcaatctctaagactgccggctttcgaaccctgccaatgccgctgtacgagaatggcacgatgaaatgcgttaccgggtttaccataacccttgaaggggccgtgccatttgacatggtagcttatggtcgaaacctgatgctgaagggttcggcaggttcctttccaacaatcgacttgctctacgactacagaccgttttttgaccaatgttccgatagtggacggatcggcttctttccggaggatgttcctaagccgaaagtggcggtcattggcgctggcatttccggactcgtggtggcaaacgaactgcttcatgctggggtagacgatgttacaatatatgaagcaagtgatcgtgttggaggcaagctttggtcacatgctttcagggacgctcctagtgtcgtggccgaaatgggggcgatgcgatttcctcctgctgcattctgcttgtttttcttcctcgagcgttacggcctgtcttcgatgaggccgttcccaaatcccggcacagtcgacacttacttggtctaccaaggcgtccaatacatgtggaaagccgggcagctgccaccgaagctgttccatcgcgtttacaacggttggcgtgcgttcttgaaggacggtttctatgagcgagatattgtgttggcttcgcctgtcgctattactcaggccttgaaatcaggagacattaggtgggctcatgactcctggcaaatttggctgaaccgtttcgggagggagtccttctcttcagggatagagaggatctttctgggcacacatcctcctggtggtgaaacatggagttttcctcatgattgggacctattcaagctaatgggaataggatctggcgggtttggtccagtttttgaaagcgggtttattgagatcctccgcttggtcatcaacggatatgaagaaaatcagcggatgtgccctgaaggaatctcagaacttccacgtcggatcgcatctgaagtggttaacggtgtgtctgtgagccagcgcatatgccatgttcaagtcagggcgattcagaaggaaaagacaaaaataaagataaggcttaagagcgggatatctgaactttatgataaggtggtggtcacatctggactcgcaaatatccaactcaggcattgcctgacatgcgataccaatatttttcaggcaccagtgaaccaagcggttgataacagccatatgacaggatcgtcaaaactcttcctgatgactgaacgaaaattctggttagaccatatcctcccgtcttgtgtcctcatggacgggatcgcaaaagcagtgtattgcctggactatgagccgcaggatccgaatggtaaaggtctagtgctcatcagttatacatgggaggacgactcccacaagctgttggcggtccccgacaaaaaagagcgattatgtctgctgcgggacgcaatttcgagatctttcccggcgtttgcccagcacctatttcctgccggcgctgattacgaccaaaatgttattcaacatgattggcttacagacgagaatgccgggggagctttcaaactcaaccggcgtggtgaggatttttattctgaagaacttttctttcaagcactggacacggctaatgataccggagtttacttggcgggttgcagttgttccttcacaggtggatgggtggagggtgctattcagaccgcgtgtaacgccgtctgtgcaattatccacaattgtggaggcattttggcaaagggcaatcctctcgaacactcttggaagagatataactaccgcactagaaattagaattc

SEQ ID NO: 13 - DNA, 2789 bp, Artificial Sequence
  FBP7iaaM gene sequence
cgaagcttcatcaaactaaattaatactctggaatattagcatgcgtgaatgtcgtcgtttgtcaattgaacttcactaatagtgttaaacaatagatacagtgataaagaatagatacagtgatacaaaatcaggtcatcctgctaatcacactacactaatagtgtaaataattaaactaaagatttaaatagaagagggggcatattagtcaagttgaaagcaagtaacacgctttgcaatttttggtgaaattctggtctgcagtaaagcagcgtacaaggccacgtctggtttatatctggaaaaaggccatttcaatgtcattttcttggccagctctttctttcctttaattttaagtgatagttaaagctagggtaggtatttataaggaaagaaagacattttaagtattgagtttttcctcattcattagtgttgtaattccaagactacaaagtctttggaagtacgtgtgaggtttctgaaaggctttgaagtcgacgggtaccatgtcaccttcacctctccttgataaccagtgcgatcatctcccaaccaaaatggtggatctgacaatggtcgataaggcggatgaattggaccgcagggtttccgatgccttcttagaacgagaagcttctaggggaaggaggattactcaaatctccaccgagtgcagcgctgggttagcttgcaaaaggctggccgatggtcgcttccccgagatctcagctggtggaaaggtagcagttctctccgcttatatctatattggcaaagaaattctggggcggatacttgaatcgaaaccttgggcgcgggcaacagtgagtggtctcgttgccatcgacttggcaccattttgcatggatttctccgaagcacaactaatccaagccctgtttttgctgagcggtaaaagatgtgcaccgattgatcttagtcatttcgtggccatttcaatctctaagactgccggctttcgaaccctgccaatgccgctgtacgagaatggcacgatgaaatgcgttaccgggtttaccataacccttgaaggggccgtgccatttgacatggtagcttatggtcgaaacctgatgctgaagggttcggcaggttcctttccaacaatcgacttgctctacgactacagaccgttttttgaccaatgttccgatagtggacggatcggcttctttccggaggatgttcctaagccgaaagtggcggtcattggcgctggcatttccggactcgtggtggcaaacgaactgcttcatgctggggtagacgatgttacaatatatgaagcaagtgatcgtgttggaggcaagctttggtcacatgctttcagggacgctcctagtgtcgtggccgaaatgggggcgatgcgatttcctcctgctgcattctgcttgtttttcttcctcgagcgttacggcctgtcttcgatgaggccgttcccaaatcccggcacagtcgacacttacttggtctaccaaggcgtccaatacatgtggaaagccgggcagctgccaccgaagctgttccatcgcgtttacaacggttggcgtgcgttcttgaaggacggtttctatgagcgagatattgtgttggcttcgcctgtcgctattactcaggccttgaaatcaggagacattaggtgggctcatgactcctggcaaatttggctgaaccgtttcgggagggagtccttctcttcagggatagagaggatctttctgggcacacatcctcctggtggtgaaacatggagttttcctcatgattgggacctattcaagctaatgggaataggatctggcgggtttggtccagtttttgaaagcgggtttattgagatcctccgcttggtcatcaacggatatgaagaaaatcagcggatgtgccctgaaggaatctcagaacttccacgtcggatcgcatctgaagtggttaacggtgtgtctgtgagccagcgcatatgccatgttcaagtcagggcgattcagaaggaaaagacaaaaataaagataaggcttaagagcgggatatctgaactttatgataaggtggtggtcacatctggactcgcaaatatccaactcaggcattgcctgacatgcgataccaatatttttcaggcaccagtgaaccaagcggttgataacagccatatgacaggatcgtcaaaactcttcctgatgactgaacgaaaattctggttagaccatatcctcccgtcttgtgtcctcatggacgggatcgcaaaagcagtgtattgcctggactatgagccgcaggatccgaatggtaaaggtctagtgctcatcagttatacatgggaggacgactcccacaagctgttggcggtccccgacaaaaaagagcgattatgtctgctgcgggacgcaatttcgagatctttcccggcgtttgcccagcacctatttcctgccggcgctgattacgaccaaaatgttattcaacatgattggcttacagacgagaatgccgggggagctttcaaactcaaccggcgtggtgaggatttttattctgaagaacttttctttcaagcactggacacggctaatgataccggagtttacttggcgggttgcagttgttccttcacaggtggatgggtggagggtgctattcagaccgcgtgtaacgccgtctgtgcaattatccacaattgtggaggcattttggcaaagggcaatcctctcgaacactcttggaagagatataactaccgcactagaaattagaattc

SEQ ID NO: 14 - DNA, 21 bp, Artificial Sequence
  Synthetic
gaaaatggcctccgatcagac

SEQ ID NO: 15 - DNA, 21 bp, Artificial Sequence
  Synthetic
cataaggggcttggaggaagt

SEQ ID NO: 16 - DNA, 20 bp, Artificial Sequence
  Synthetic
gaagcctcatcgataccgtc

SEQ ID NO: 17 - DNA, 19 bp, Artificial Sequence
  Synthetic
ctaccactaccatcatggc

(19) United States
(12) Patent Application Publication
(10) Pub. No.: US 2011/0145947 A1
(43) Pub. Date: Jun. 16, 2011

(54) Plant Expression Vector Expressing Auxin Synthesis Related Gene and the Use Thereof in Improving Cotton Fiber Trait

(75) Inventors: Yan Pei, Chongqing (CN); Lei Hou, Chongqing (CN); Demou Li, Chongqing (CN); Shuiqing Song, Chongqing (CN); Xianbi Li, Chongqing (CN); Ming Luo, Chongqing (CN); Yuehua Xiao, Chongqing (CN); Xuelian Zheng, Chongqing (CN); Qiwei Zeng, Chongqing (CN); Mi Zhang, Chongqing (CN); Kun Qiu, Chongqing (CN); Fengtao Luo, Chongqing (CN)

(73) Assignee: SOUTHWEST UNIVERSITY, Chongqing (CN)

(21) Appl. No.: 13/055,586

(22) PCT Filed: Jan. 22, 2009
(86) PCT No.: PCT/CN2009/000095

§ 371 Date: Jan. 24, 2011

(30) Foreign Application Priority Data
Jul. 25, 2008 (CN) 200810142518.3
Jan. 22, 2009 (CN) PCT/CN2009/000095

(51) Int. Cl. A01H 1/00; C12N 15/63; C12N 1/00; C12N 15/29

(52) U.S. Cl. 800/287; 435/320.1; 435/243; 536/23.6

(57) ABSTRACT

A method of expressing auxin synthetase gene specifically in cotton seed coat and fiber, which comprises constructing plant expression vector capable of expressing auxin synthetase gene specifically by fusing a tissue-specific promoter with an auxin synthetase gene, and then integrating the construct into a cotton genome. The method can significantly improve the yield and the quality of cotton fiber, thereby providing fiber with high quality for textile industry.

### CROSS-REFERENCE TO RELATED APPLICATIONS

The present application is a Section 371 U.S. national stage entry of pending International Patent Application No. PCT/CN2009/000095, International Filing Date, Jan. 22, 2009, which claims priority to Chinese Patent Application No. 200810142518.3, filed Jul. 25, 2008, the contents of which are incorporated by reference in their entireties.

### TECHNICAL FIELD

The invention relates to a plant expression vector and use thereof, especially to a plant expression vector expressing auxin synthesis related gene and use thereof in improving cotton fiber trait.

### BACKGROUND ART

Cotton is the most important natural fiber crop as well as the most important industrial crop in the world. China is the biggest country of textile production and consumption in the world, where the cotton industry plays a significant role in national economy. Recently, with the increasing living standard of people and the development of textile technologies, the demand on cotton fiber quality also rises. Especially, the technology revolution of replacing ring spinning with rotor spinning in recent years requires fibers which are longer, more tenacious, finer, and more uniform. However, the popular varieties of cotton at present mostly provide low fiber quality, single length, low fiber strength and rough fibers. The up-market varieties of cotton yarns with the count of more than 60 are scarce, far more than being enough for meeting the market demands. This directly causes that raw cotton is less competitive in the international market. Meanwhile, cotton production thereby is stuck in the dilemma of structural conflictions in the recent years: on the one hand, the yield of raw cotton is decreasing year by year while the inventory raw cotton continues to increase, leading to the great backlog of capital; on the other hand, the amount of import cotton keeps on going up. Whether the fiber yield and quality of the cotton varieties can be improved rapidly directly decides the fate of the cotton industry and the existence and development of the production and process industry of textiles.

The yield and quality traits of cotton are the quantitative traits controlled by multiple genes, and the yield trait and the quality trait correlate to each other negatively genetically. The cultured varieties of cotton are chiefly those of Gossypium hirsutum, while the genes for excellent fiber quality are mainly derived from diplont Gossypium thurberi (fiber strength), Gossypium anornalurn (fiber strength and fineness) and tetraplont Gossypium barbadense (fiber strength and fineness) etc. The applications of these genes with excellent traits are limited by many factors in the conventional breeding. Cotton yield cannot be significantly increased only by the current cotton genetic germplasm resources and the conventional breeding means, and the demands of the rapidly developing textile technology revolution on fiber quality cannot be satisfied. Breeding with genetic engineering technologies can break through the genetic barriers among species and achieve the directional transfer of the excellent target genes, which is advantageous in terms of descendants tending to be stable and short breading cycle. This provides the new route of improving cotton fiber yield and quality. However, at present, genes directly relevant to the formation, yield and quality (strength, fineness and length etc.) of cotton fibers have not been obtained, so there are few effective target genes for improving cotton fibers by genetic engineering. The molecular mechanisms of the generation, development and the formation of quality of cotton fibers have not been revealed well. All of these factors greatly impede the course of improving yield and quality of cotton fibers.

The current researches indicate that cotton fiber is the unicellular fiber developing from four courses: the differentiation initiation of the epidermal cells of outer integument of cotton ovule, elongation, thickening (secondary wall synthesis) and the maturation dehydration. The final length of cotton fiber cells can be up to 20-30 mm, or higher, up to 35-40 mm, and the ratio of length to diameter thereof reaches 1,000-3,000. Such a high ratio of length to diameter is the result of intense elongation of fiber cells, in which there must be the involvement of the plant hormone promoting cell growth and elongation. The initiation and elongation of fiber cells are both closely related to auxin (e.g. indole-3-acetic acid, IAA). Making use of the cotton ovule cultured in vitro, people found that cultured unfertilized ovules could not produce fibers, but adding gibberellin (GA) and IAA to the culture medium could induce growth of fiber; auxin antagonist treatment showed that auxin was a key factor for the elongation of the fiber primordium (Beasley C A, 1973, Science, 179: 1003-1005; Beasley C A, et al., 1973, American J Bot, 60, 130-139). Giavalis et al. reported in 2001 the effects of GA₃ (gibberellin A3) and IAA treatment on the number of fibers of the cultured unfertilized ovules. The results showed that the application of exogenous GA and IAA before or after flowering could increase significantly the number of the cultured unfertilized ovule fibers (Giavalis S., et al., 2001, J Cotton Sci., 5, 252-258). Seagull and Giavalis further found in 2004 that in the natural growth state, the GA₃ and IAA treatment of cotton bud or boll could significantly increase the number of fiber cells. Moreover, the IAA treatment of the cotton boll before flowering or after flowering could increase the number of fiber cells of a single cotton seed by 58% (Seagull R W, etc., 2004, J Cotton Sci., 8, 105-111). These studies have shown that IAA can promote the production of cotton fibers, and is closely related to fiber development and growth.

Although the application of exogenous auxin has a good effect, it is often difficult to achieve the application in production: the application of auxin one by one to the flowers or buds and bolls results in the very large workload, high labor costs, and the difficulty in popularizing the application on a large scale. Moreover, the extensive use of auxin not only increases the costs of production but also cause environmental pollution. In contrast, by controlling auxin biosynthesis enzyme genes, the regulation of auxin levels in specific organs from the endogenous perspective to promote the development of organs to be harvested is a very effective strategy. This strategy has at least the following several advantages: 1) high efficiency and low costs, because once the auxin biosynthesis enzyme genes are introduced into plants, no external application of auxin or other treatment is needed. Moreover, the exogenously applied auxin goes into the cell by diffusion, while the endogenous hormone is generated from inside the cell. Thus, the effect of the transgenic endogenous control of auxin is often better than the exogenous application; 2) a small negative impact on crops—owing to the low action concentration of auxin, the excessively low or high auxin concentration both will bring adverse effects on plant development. The endogenous expression of auxin synthetase gene, under the suitable circumstances for expression levels and expression sites, can exert effect only on specific target organs (tissues), without affecting the normal development of other parts of the plant; 3) compared with the application of exogenous auxin and the artificial synthetic production of regulators, the endogenous regulation of auxin synthetase genes results in little environmental pollution and little harm to human health (Li Y, et al., 2004, Transgenics of plant hormones and their potential application in horticultural crops. In: Genetically Modified Crops: their development, uses, and risks. New York: Food Products Press, 101-112).

However, the strategy of using auxin synthetase genes for increasing yield and improving quality is not successful in terms of cotton breeding. In 1999, John M E placed the two enzyme genes iaaM and iaaH relating to the biosynthesis of auxin IAA under fiber-specific promoter E6, which were introduced into Gossypium hirsutum DP50 by Agrobacterium-mediated method. It was thereby found that the IAA content was increased by 2 to 8 times in most of the transgenic lines. However, the fiber length, fineness and strength were not distinctly different from those of the wild types (Basra A S et al., 1999, Cotton Fiber, New York: Food Products Press, 271-292). Up to now, there is no report on the successful improvement of cotton fiber quality by the endogenous expression of hormone biosynthesis enzyme gene. It is generally doubted that cotton fiber quality can be improved by the hormone biosynthesis enzyme gene.

### SUMMARY OF INVENTION

The technical problem to be solved by this invention is to provide a plant expression vector expressing auxin synthesis related gene and use of the plant expression vector in improving cotton fiber trait for solving the problem for improving the cotton fiber yield and quality which the present methods of endogenous expression of plant auxin synthetase genes fail to solve.

The present application further provides the use of the plant expression vector of this invention in improving cotton fiber trait.

The invention further provides a method of producing a transgenic plant comprising the plant expression vector according to the invention.

According to one aspect of the invention, the plant expression vector according to the invention at least comprises a nucleotide sequence expressing auxin synthesis related gene and consisting of a plant auxin synthetase gene and a plant seed coat-specific promoter, wherein the nucleotide sequence is constructed by operably linking the gene encoding plant auxin synthetase and the gene encoding plant seed coat-specific promoter. The preferable plant auxin synthetase gene is Agrobacterium tumefaciens tms (tumour morphology shooty) gene (usually referred to as iaaM gene); the preferable plant seed coat-specific promoter is FBP7 (Floral Binding Protein 7) gene promoter. Thus, the preferable nucleotide sequence encoding and expressing auxin synthesis related gene is the nucleotide sequence having the sequence represented by SEQ ID NO: 13. After the nucleotide expressing auxin synthesis related gene is obtained, it is inserted into the expression vector for constructing the plant expression vector expressing auxin synthesis related gene of this invention. The preferable plant expression vector has the vector structure as shown in FIG. 2. During the process of constructing the plant expression vector, for the convenience of gene operation, a part of variable non-encoding sequence is present between FBP7 promoter and iaaM gene. Moreover, during the process of placing the FBP7 promoter upstream of the 5′ end of iaaM gene by means of gene operation, the uses of different cloning means will produce different non-encoding sequences. However, the linking of FBP7 promoter and iaaM gene and the biological function of FBP7 promoter and iaaM gene together are not affected thereby. Thus, as long as the FBP7 promoter is located upstream of the 5′ end of iaaM gene and the expression of iaaM gene is promoted, whatever non-encoding sequences are present between them, they fall within the scope of the present patent.

According to another aspect of the invention, a transformant is provided, which is obtained by transfecting a host with the plant expression vector of the invention. The transformant can be used for transforming plants to obtain transgenic plants.

According to a further aspect of this invention, the use of the plant expression vector of this invention in improving cotton fiber trait is provided. The object of improving cotton fiber trait is achieved by expressing auxin synthesis related gene constructed herein in plants and thereby modulating the level of auxin.

According to a further aspect of this invention, a method of producing a transgenic plant is provided. A plant (cotton) is transformed by the transformant above of this invention to obtain a transgenic plant.

Specifically, the method of improving cotton fiber trait comprises the following several steps: 1) obtaining a seed coat and fiber-specific expression promoter; 2) obtaining an auxin synthesis related gene; 3) fusing the specific promoter obtained by separation and cloning in step 1) with the auxin synthesis related gene obtained by separation and cloning in step 2) to construct the plant expression vector specifically expressing the auxin synthesis related gene; 4) integrating the plant expression vector specifically expressing the auxin synthesis related gene obtained in step 3) into a cotton genome; 5) further culturing and cultivating the cotton obtained in step 4) and thereby obtaining the transgenic cotton plant.

Wherein, the specific expression promoter as described in step 1) can be a natural promoter isolated and cloned from animals, plants or micro-organisms, or a promoter artificially modified or designed and synthesized.

Wherein, the plant auxin synthesis related gene as described in step 2) can be a natural gene isolated and cloned from animals, plants or micro-organisms, or a gene artificially modified or designed.

Wherein, the method as described in step 3) of fusing the specific promoter with the auxin synthesis related gene to construct the expression vector specifically expressing the auxin synthesis related gene is the conventional method in the art, and the used vector is the conventional vector used in the field of plant transgene.

Wherein, the used method as described in step 4) of integrating the expression vector into the cotton genome is the conventional method of plant transgene, e.g. the Agrobacterium mediated method or gene gun bombardment.

Preferably, the specific promoter above is the ovary-specific promoter or the seed coat-specific promoter or seed coat and fiber-specific promoter. More preferably, the above-mentioned types of seed coat and fiber-specific promoter is FBP7 (Floral Binding Protein 7) gene promoter, the ovary-specific promoter above is AGL5 (Agamous Like protein 5) gene promoter, and the fiber-specific promoter above is E6 gene promoter.

Preferably, the plant hormone synthesis related gene above is auxin synthesis related gene. More preferably, the plant hormone synthesis related gene of the present invention is Agrobacterium tumefaciens tms (tumour morphology shooty) gene (often referred to as iaaM gene).

Within the present invention, “cotton fiber trait” refers to the quantity and quality trait of cotton fiber, including the quantity, length, fineness, strength, uniformity and so on of fiber.

Within the present invention, “transgenic cotton” refers to the cotton into which, through molecular biology, biotechnology means, a gene of other organism are transferred, so that the genetic material in the modified cotton is modified. Gene for modification can be derived from plants, animals and microorganisms, or can be artificially synthesized and modified.

The term “lint percentage” of the present invention refers to the ratio of the weight of fibers on seed cotton to the weight of seed cotton, expressed in percentage. That is, the proportion of the weight of fibers in the total weight of the seed and the fibers.

Within the invention, “fiber strength” refers to the greatest load that a bundle of fibers can bear when stretched to the extent of being about to break, which is indicated by cN/tex. Tex is the weight in gram of fiber of 1000 meters.

By a large amount of researches and analysis as well as early experiments, it is thought in this invention that although John et al. did not succeed, this does not mean that the strategy of using the plant hormone biosynthesis enzyme gene to increase cotton yield and improve fiber quality cannot be successful. That's because: now that the exogenous application of plant hormones like IAA etc. has the notable effects of increasing the number of cotton fiber cells and improving fiber quality, it is feasible to modulate the hormone amount from the endogenous perspective by controlling the plant hormone synthetase gene so as to promote cotton fiber development. The reason that the prior researches do not result in the expected effects lies in: the suitable promoter is not found. Thus, cotton fiber growth and development can only be effectively influenced to obtain the expected effects if: the suitable promoter is chosen; the expression of the plant hormone synthesis related gene is controlled with the proper intensity at the particular part of cotton and at the particular time of development; the action concentration, time and part of the plant hormone in vivo is precisely regulated. However, there are various types of promoters, so it is impossible to predict which type of promoter linked with the auxin related gene is effective on cotton fibers. In accordance with the features of cotton fiber development, on the basis of screening for promoters on a large scale, the inventors inventively uses a seed coat-specific promoter, FBP7 (Floral Binding Protein 7) gene promoter (derived from petunia), an ovary-specific promoter, AGL5 (Agamous Like protein 5) gene promoter (derived from Arabidopsis) and a fiber-specific promoter, E6 gene promoter (derived from Gossypium hirsutum) as the primary elements to construct the new gene expression vector and establishes a set of adaptable methods of improving cotton fiber trait.

Under the condition that the promoter and the target gene are determined, the manner of the method of this invention of fusing the promoter with the target gene can be one conventional method in the art, and the fused new gene can be transferred into the vector conventional in the art to construct the expression vector which is then transferred into cotton. Obviously, the expression vector above can be constructed as a monovalent vector containing a single gene or as a bivalent or trivalent vector containing multiple genes, or as other types of vectors. In the process of using the method of modifying cotton of the present invention, either a target gene is expressed only at one part or multiple genes are expressed in multiple parts and multiple developmental stages. The method of the present invention has provided technical solutions for them.

The method of the invention for improving cotton fiber trait is to regulate the expression of auxin synthetase by specifically expressing an auxin synthesis related gene at the seed coat and fiber of cotton, and control the development of cotton seeds and the initiation of fiber development and elongation by the endogenous modulation of the amount of the corresponding hormone in the particular tissue organ of cotton, thereby achieving the object of improving cotton fiber yield and quality (length, fineness and strength). The results demonstrated that the number of cotton fibers, the traits of which are improved by the method of the invention, is significantly increased, and the yield thereof is notably raised; the quality of cotton fibers is remarkably improved; the number of seeds thereof is increased and the lint percentage is raised significantly. The method of the invention is simple and can be easily carried out with the significant effects, which brings high-yield, high-quality fiber raw materials to the textile industry and results in enormous economic benefits.

### DESCRIPTION OF DRAWINGS

FIG. 1: The flow chart of constructing the expression vector of auxin synthesis gene under the regulation of the specific promoter (including FBP7, AGL5, and E6).

Km, kanamycin resistance gene; Amp, ampicillin resistance gene; NPTII, neomycin phosphotransferase gene; GUS, β-glucuronidase gene; 35S, plant constitutional promoter derived from cauliflower mosaic virus; Pnos, opine synthetase gene promoter; nos, opine synthetase gene terminator; LB, T-DNA left boundary; RB, T-DNA right boundary. The backbone vector used to construct plant expression vector is the P5 vector modified on the basis of pBI121, comprising the GUS gene under the control of CaMV 35S promoter, which facilitates screening for GUS staining for transformants in the course of plant genetic transformation.

FIG. 2: the structural diagram of the plant expression vector of the invention containing the specific promoter FBP7.

FIG. 3: the southern analysis of auxin synthetase gene iaaM in transgenic cotton.

A, genomic DNAs of 11 lines of p5-FBP7: iaaM transgenic cotton were cleaved by XbaI, and then they underwent southern hybridization with the iaaM gene fragments. Hybridization fragments of different sizes were obtained in different lines. There is no hybridization signal in the wild-type control. 1, 2, 6 . . . 20 are the different transgenic lines of FBP7-iaaM; WT is the wild-type control.

B, genomic DNAs of 6 lines of p5-E6: iaaM transgenic cotton were cleaved by XbaI, and then they underwent southern hybridization with the iaaM gene fragments. Hybridization fragments of different sizes were obtained in different lines. There is no hybridization signal in the wild-type control. 1, 2, 5, 8, 10 and 11 are respectively the different transgenic lines of p5-E6-iaaM (IE1-1, IE1-2, IE1-5, IE1-8, 1E1-10 and 1E1-11); WT is the wild-type control.

C, genomic DNAs of 8 lines of p5-AGL5-iaaM transgenic cotton were cleaved by XbaI, and then they underwent southern hybridization with the iaaM gene fragments. 3, 4, 6, 7, 10, 11, 12 and 14 are respectively the different transgenic lines of p5-AGL5-iaaM cotton (IG1-3, IG1-4, IG1-6, IG1-7, IG1-10, IG1-11, IG 1-12 and IG1-14); WT is the wild-type control, and there is no hybridization signal in the wild-type control.

FIG. 4: the RT-PCR analysis of the specifically expressed plant auxin synthetase gene iaaM in the FBP7-iaaM transgenic cotton.

A, the analysis of the expression of iaaM in the 11 lines of FBP7-iaaM transgenic cotton and the wild type. The expression of iaaM was detected in three lines, namely 9^{#}, 14^{#}, 20^{#}. Wherein, the expression in 9^{#} was the strongest, that in 14^{#} was intermediate, and that in 20^{#} was the weakest. 1, 2, 6, 7, 9, 10, 11, 14, 15, 18, 20: numbers of different lines. B, in the different developmental stages of the ovule and fiber of FBP7-iaaM transgenic cotton 9^{#}, expression of iaaM gradually weaken as time went on, and the expression of iaaM basically cannot be detected after 15 days; −2:−2 dpa, the materials of two days before flowering; 0, 1, 2, 3, 5, 10, 15, 20, 30:0 dap, 1 dpa . . . 30 dpa, the materials of different days after flowering. The upper part: the RT-PCR results of iaaM gene, the amplification products of the specific primers of iaaM gene (SEQ ID NOs: 9 and 10), amplified for 35 cycles. The middle part: the RT-PCR results of GhHis gene; the amplification products of the specific primers of Histone (SEQ ID NOs: 7 and 8), amplified for 35 cycles. The lower part: the RT-PCR results of iaaM with RNA as the template, showing that there is no DNA contamination which can be detected in the used RNA. Control: the separate negative plant as control; P: the positive control with pUC-iaaM plasmid as the template.

FIG. 5: the RT-PCR analysis of the specifically expressed plant auxin synthetase gene iaaM in the E6: iaaM transgenic cotton.

The analysis of the expression of iaaM in the 11 different lines of E6: iaaM transgenic cotton and the wild type. The rather high expression level of iaaM was detected in line 11^{#}. The expression of iaaM gene was also detected in lines 1^{#}, 2^{#}, 8^{#}, 10⁴, 14^{#} and 17^{#}. 1, 2, 5, 8, 10, 11, 13, 14, 17, 19, 21: numbers of different transgenic lines. The upper part: the RT-PCR results of iaaM gene, the amplification products of the specific primers of iaaM gene (SEQ ID NOs: 9 and 10), amplified for 35 cycles. The middle part: the RT-PCR results of GhHis gene; the amplification products of the specific primers of Histone (SEQ ID NOs: 7 and 8), amplified for 35 cycles. The lower part: the RT-PCR results of iaaM with RNA as the template, showing that there is no DNA contamination which can be detected in the used RNA. Control: the separate negative plant as control.

FIG. 6: the RT-PCR analysis of the specifically expressed plant auxin synthetase gene iaaM in the Ag15: iaaM transgenic cotton.

The analysis of the expression of iaaM in the 11 different lines of Ag15-iaaM transgenic cotton and the wild type. The rather high expression level of iaaM gene was detected in three lines: 6^{#}, 7⁴, 10⁴. Secondly, the 15⁴, 23^{#} transgenic lines. The expression of iaaM gene was also present in 2^{#}, 3⁴, 4⁴, 16^{#}, 17^{#} and 21^{#}. 2, 3, 4, 6, 7, 10, 15, 16, 17, 21, 23: numbers of different lines. The upper part: the RT-PCR results of iaaM gene, the amplification products of the specific primers of iaaM gene (SEQ ID NOs: 9 and 10), amplified for 35 cycles. The middle part: the RT-PCR results of GhHis gene; the amplification products of the specific primers of Histone (SEQ ID NOs: 7 and 8), amplified for 35 cycles. The lower part: the RT-PCR results of iaaM with RNA as the template, showing that there is no DNA contamination which can be detected in the used RNA. Control: the separate negative plant as control.

FIG. 7: the Real-time PCR analysis of iaaM gene in the ovule of FBP7-iaaM transgenic cotton.

The expression degrees of iaaM gene in 11 transgenic lines were varied; the expression amount in 9^{#}, 14^{#} was high (A); the expression level of iaaM gene in cotton ovule and fiber decreased gradually from two days before flowering to 10 dpa, peak appeared on 0 dap, and iaaM gene expression was undetectable on day 15 and later, (B). Control: the separate negative plant as control.

FIG. 8: the comparison of the amount of free IAA in the ovule and fiber of the FBP7/E6/AGL5-iaaM transgenic cotton with that of the control.

The amounts of free IAA in cotton ovule one day (1d) before flowering to 5 days (5d) after flowering were measured. It was found that the amounts of free IAA in E6-iaaM and AGL5-iaaM transgenic cotton ovules were not significantly changed compared with the control; while the amount of free IAA in FBP7-iaaM transgenic plant was significantly increased compared with the control, about 2-8 times higher than that of the control. Wherein, the test sample was the mixed extract of ovule and fiber. Control: the separate negative plant as control. Materials were taken repeatedly at each time point for 3 times, and the average value was taken for diagram analysis.

FIG. 9: the comparative scanning electron microscopy diagram of surface of cotton ovule of FBP7-iaaM transgenic cotton and the wild-type cotton.

A, the wild-type ovule surface on the flowering day, showing the initial fibers, with the amplification of 70 times; B, the ovule surface of the transgenic cotton transformed by iaaM under the control of specific promoter FBP7, showing the initial fibers, with the amplification of 70 times; C, the further amplification of FIG. A, showing the shape and number of initial fibers, with the amplification of 500 times; D, the further amplification of FIG. B, showing the shape and number of initial fibers, with the amplification of 500 times; the initial fiber distribution in D was significantly more concentrated than that in C, and the number was greater. In C, D, Bar=10 μm.

FIG. 10: the comparative scanning electron microscopy diagram of surface of cotton ovule of E6-iaaM transgenic cotton and the wild-type cotton.

A, the wild-type ovule surface on the flowering day, showing the initial fibers, with the amplification of 80 times; B, the further amplification of FIG. A, showing the shape and number of initial fibers, with the amplification of 500 times; C, the ovule surface of the transgenic cotton transformed by iaaM under the control of specific promoter E6, showing the initial fibers, with the amplification of 80 times; D, the further amplification of FIG. C, showing the shape and number of initial fibers, with the amplification of 500 times.

FIG. 11: the comparative scanning electron microscopy diagram of surface of cotton ovule of AGL5-iaaM transgenic cotton and the wild-type cotton.

A, the wild-type ovule surface on the flowering day, showing the initial fibers, with the amplification of 80 times; B, the further amplification of FIG. A, showing the shape and number of initial fibers, with the amplification of 500 times; C, the ovule surface of the transgenic cotton transformed by iaaM under the control of specific promoter AGL5, showing the initial fibers, with the amplification of 80 times; D, the further amplification of FIG. C, showing the shape and number of initial fibers, with the amplification of 500 times.

FIG. 12: the microscopic observation of tissue sections of FBP7-iaaM transgenic cotton ovule and fiber.

The protrusions of the fiber primordia were obviously visible on the ovule surface of FBP7-iaaM transgenic cotton of 0 dpa; the growth of the fiber primordia on the ovule surface of transgenic cotton of 1 dpa was more prominent than that of the control; on the transgenic ovule surface of 2 dpa, the fibers already remarkably grew and the number of fibers was greater than that of the control. Control: the separate negative plant as control. FBP7-iaaM represents FBP7-iaaM transgenic cotton; 0 dpa represents ovule on the flowering day; 1 dpa represents the ovule one day after flowering; 2 dpa represents the ovule two days after flowering; A, D, E, H, I, J, amplification of 10 times, Bar=5 μm; B, C, F, G, amplification of 40 times, Bar=2 μm.

FIG. 13: the statistic results of the early FBP7-iaaM transgenic cotton fibers.

The number of the fibers on ovule two days after flowering of transgenic plants was increased obviously in contrast to the control. The number of fibers on the ovule surface of 9^{#}, 14^{#} FBP7-iaaM transgenic cotton lines of 2 dpa was around 6000, the average number of the control was 5940. The numbers of fibers of the two lines 9^{#}, 14^{#} were significantly increased compared with the wild type, wherein the number was increased by about 11.3% in 9^{#}, and in 14^{#}, 15.1%.

FIG. 14: the comparison of the seed size and the amount of fuzz of FBP7-iaaM transgenic cotton and the control.

A, Compared with the control, the seed size of transgenic cotton was significantly reduced, and the amount of fuzz was decreased. B, As analyzed through delinting by sulfric acid, the amount of fuzz of the transgenic cotton was decreased by about 10% as compared with the control. Control: the separate negative plant as control. FBP7-iaaM represents FBP7-iaaM transgenic cotton.

### SPECIFIC EMBODIMENTS

Further description will be made in details in combination with the accompanying figures. However, the following description is not intended to limit the invention. Any deformation and changes to the present invention should be covered by the scope defined by the appended claims without departing from the spirit of the invention.

The reagents and drugs in the examples of this invention are all available commercially unless indicated otherwise. The materials and methods are in accordance with “Molecular Cloning: A Laboratory Manual” (Sambrook and Russell, 2001) unless indicated otherwise.

### Example 1

### The Preparation of Cotton Genome

### 1. Extracting DNA

0.5-1.0 g of young cotton leaves were selected and quickly ground into powder in liquid nitrogen. Add 3 mL CTAB extract preheated at 65° C. (100 mmol/L Tris-HCl (pH8.0), 20 mmol/L EDTA (pH8.0), 1.5 mol/L NaCl, 2% CTAB (W/V), 4% PVP40 (W/V) and 2% mercaptoethanol (V/V), PVP and mercaptoethanol were added before use), agitate and mix fastly; water bath at 65° C. for 30 mins, then add 1 mL 5 mol/L KAc, ice bath for 20 mins, extract once by using the equal volume of chloroform: isoamyl alcohol (24:1) (centrifugate at 10,000 r/min at 4° C. for 5 mins), take supernatant, add ⅔× volume of −20° C. pre-cooled isopropanol, mix and stand for about 30 mins, pick out flocculent precipitate with a glass rod, rinse several times repeatedly with 75% ethanol, and then rinse once with absolute ethanol, blow-dry, and resuspend in 500 μL, TE. Add 1 μL, RNaseA (10 mg/mL), treat at 37° C. for 1 h. Then, extract respectively once by using phenol (pH8.0): chloroform: isoamyl alcohol (25:24:1) and chloroform: isoamyl alcohol (24:1) (10,000 r/min, centrifugate at 4° C. for 5 mins), take the supernatant and precipitate with ethanol. Wash the precipitate with 75% ethanol, air-dry, dissolve in 200 μL, TE, store at −20° C. for use.

### 2. Extracting RNA

Select about 3 g of fresh cotton material, grind quickly in liquid nitrogen into fine powder, load the powder into a 50 mL centrifuge tube, add 15 mL RNA extract preheated at 65° C. (2% CTAB (W/V), 2% PVP (W/V), 100 mmol/L Tris-HCl (pH8.0), 0.5 g/L Spermidine, 2.0 mol/L NaCl, 2% mercaptoethanol (V/V, added before use)), and mix upside down. Water bath at 65° C. for 3-10 mins, and mix 2-3 times during the water bath. Extract twice by using chloroform: isoamyl alcohol (24:1) (10,000 r/min, at room temperature for 5 mins). Take the supernatant, add ¼ volume of 10 mol/L LiCl solution, stand at 4° C. for 6 h, extract respectively once with chloroform: isoamyl alcohol (25:24:1) (10,000 r/min, at room temperature for 5 mins). Add 2× volume of absolute ethanol, precipitate in refrigerator at −70° C. for more than 30 mins. Centrifuge at 12,000 r/min at 4° C. for 20 mins, and discard the supernatant. Dissolve the precipitate with 200 μL of DEPC-treated water. Extract respectively once by using phenol (pH4.5): chloroform: isoamyl alcohol (25:24:1), chloroform: isoamyl alcohol (24:1) (10,000 r/min, at room temperature, for 5 mins). Add 1/10 volume of 3 mol/L NaAc solution and 2.5× volume of absolute ethanol, precipitate in refrigerator at −70° C. for more than 30 mins. Centrifuge at 12,000 r/min at 4° C. for 20 mins, and discard the supernatant. Rinse the precipitate with 70% alcohol once and air-dry. Add 200 μL, of DEPC-treated water to dissolve. Test the RNA quality with non-denaturing agarose gel electrophoresis and ultraviolet spectrophotometer scanning.

### 3. The PCR Amplification of the Genome Sequence

| | | |
|---|---|---|
| 10 × Ex PCR buffer (Mg²⁺ free) | 2.5 | μL |
| 2.5 mmol/L dNTPs | 2 | μL |
| 25 mmol/L MgCl₂ | 2 | μL |
| Primer 1 (5 μmol/L) | 1 | μL |
| Primer 2 (5 μmol/L) | 1 | μL |
| Ex Taq DNA polymerase | 1 | U |
| Genome DNA | about 60 | ng |

25 μL amplification system

Amplification procedures: 94° C., 5 mins; 94° C., 30 secs, 56° C., 30 secs, 72° C., 1.5 mins, 35 cycles; extension at 72° C. for 10 mins.
4. Recovering of DNA Fragments, Ligation and Transforming E. coli DH5α

Recover fragments with a length of less than 2.0 kb by centrifugation. With UV lamp, cut the agarose gel blocks containing the target fragments with a clean blade. Drill a hole with 5# needle in the bottom of a 0.5 mL centrifuge tube and fill with glass wool of the appropriate size. Put the agarose blocks containing the target fragments into the 0.5 ml centrifuge tube filled with glass wool, freeze in liquid N₂ fastly, set the frozen 0.5 ml centrifuge tube into a 1.5 mL centrifuge tube, and centrifuge at 13,000 r/min for 3 mins. Add 1/10× volume of 3 mol/L NaAc (pH5.2) and 3× volume of absolute ethanol to the effluent fluid (including DNA), mix and stand at −70° C. for 30 mins. Centrifuge at 13,000 r/min at 4° C. for 15 mins, and collect DNA precipitates, and wash the precipitates with pre-cooled 75% ethanol. Dry at room temperature. Obtain the target fragments by dissolving the precipitates with the suitable amount of TE. Quantify the recovered fragments with agarose gel electrophoresis. Recover fragments with a length of greater than 2.0 kb with a kit (Roche, Ltd).

Establish the following ligation system of the recovered fragments and pUCm-T vector (Sangon Biotech (Shanghai) Co., Ltd.):

| | |
|---|---|
| 10 × T4 DNA ligation buffer | 1 μL |
| DNA fragments of vector | 1 μL |
| DNA fragments of the exogenous ligation products | 1 μL |
| T4 DNA ligase | 1 μL |

Make up the volume with double distilled water to 10 μL, of the ligation system

The molar ratio of DNA fragments of vector to DNA fragment of the exogenous ligation product is 1:3. Ligate at 16° C. for 12 h. Then, use the ligation product to transform E. coli DH5α.

### Example 2

### The Preparation of the Nucleotide Sequence Expressing Auxin Synthesis Related Gene and the Plant Expression Vector

### 1. Obtaining the Specific Promoters

Design primers (SEQ ID NOs. 1 and 2) according to petunia seed coat-specific promoter FBP7 (GenBank accession number: U90137). Obtain a fragment of about 500 bp from the petunia genome by PCR amplification. The amplified DNA fragment was cloned into pUCm-T (Sangon Biotech (Shanghai) Co., Ltd.). The sequencing analysis showed that it was petunia FBP7 specific promoter; see SEQ ID NO. 3. The clone vector was designated as pUC-FBP7.

Design E6 specific primers (SEQ ID NOs. 4 and 5) according to cotton fiber-specific promoter E6. Obtain a fragment of about 1.4 kb from the Gossypium hirsutum genome by amplification. The amplified DNA fragment was cloned into pUCm-T (Sangon Biotech (Shanghai) Co., Ltd.). The sequencing analysis showed that it was Gossypium hirsutum E6 fiber-specific promoter; see SEQ ID NO. 6. The clone vector was designated as pUC-E6.

Design AGL5 specific primers (SEQ ID NOs. 7 and 8) according to arabidopsis seed-specific promoter AGL5(Genebank accession number: AC006931.6). Obtain a fragment of about 2.0 kb from the arabidopsis genome by amplification. The amplified DNA fragment was cloned into pUCm-T (Sangon Biotech (Shanghai) Co., Ltd.). The sequencing analysis showed that it was arabidopsis AGL5 specific promoter; see SEQ ID NO. 9. The clone vector was designated as pUC-AGL5.

2. Obtain Agrobacterium tumefaciens iaaM Gene

Design primers (see SEQ ID NOs. 10 and 11) according to Agrobacterium tumefaciens Ti plasmid tms (iaaM) gene sequence (GenBank accession number: K02554), obtain SEQ ID NO.: 12 from Agrobacterium tumefaciens Ti plasmid T-DNA by PCR amplification and by cloning into pUCm-T (Sangon Biotech (Shanghai) Co., Ltd.) and sequencing analysis. The clone vector was designated as pUC-iaaM.

### 3. Constructing the Vector Specifically Expressing Plant Hormone Synthesis Related Gene

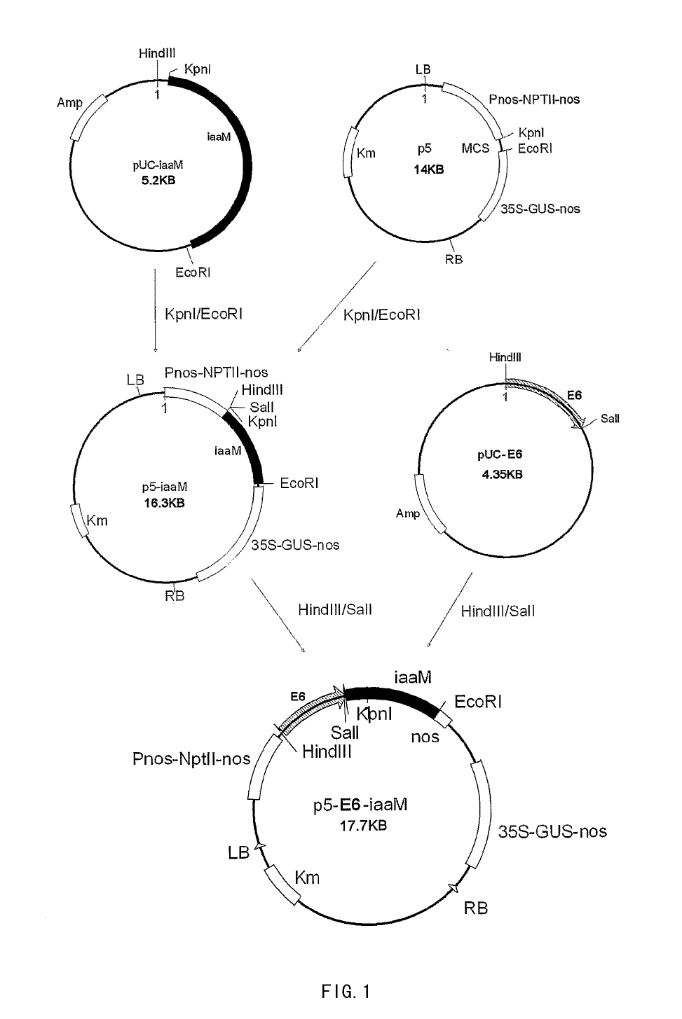

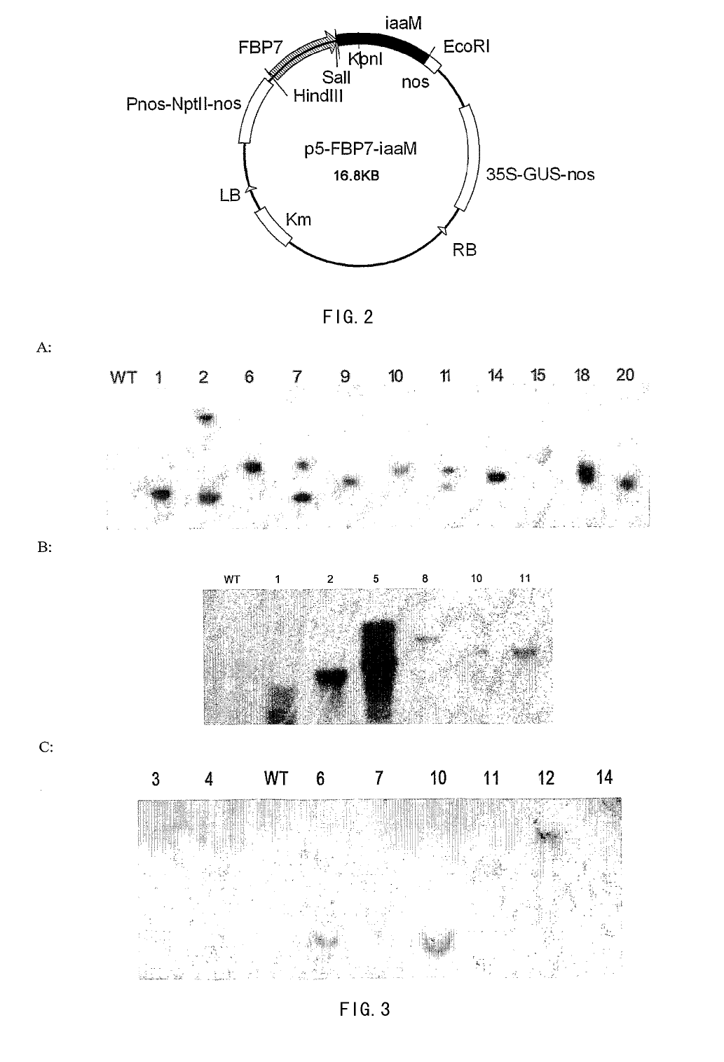

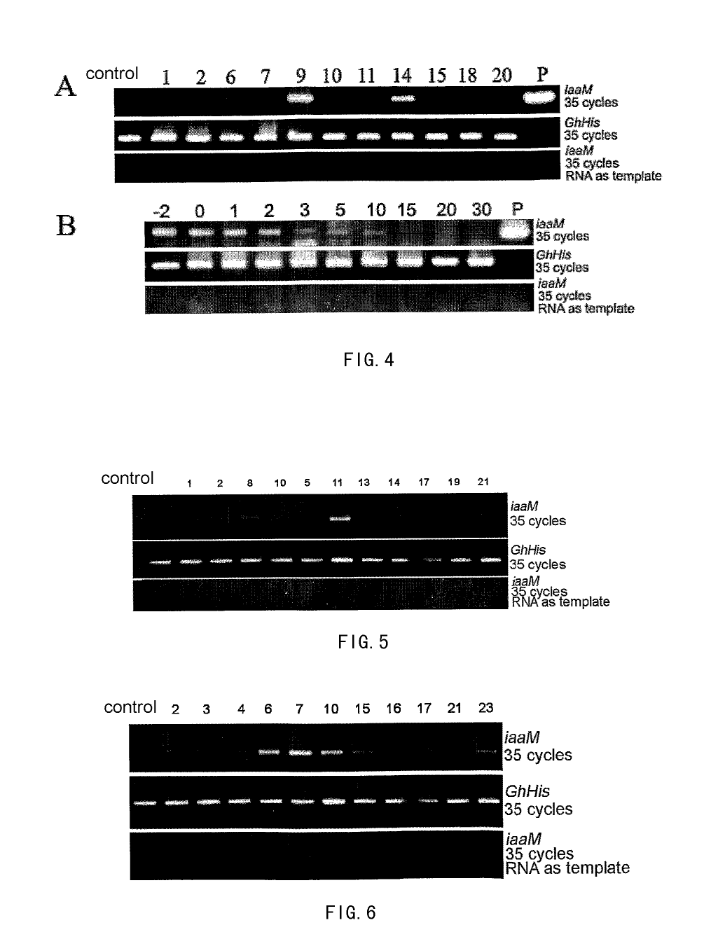

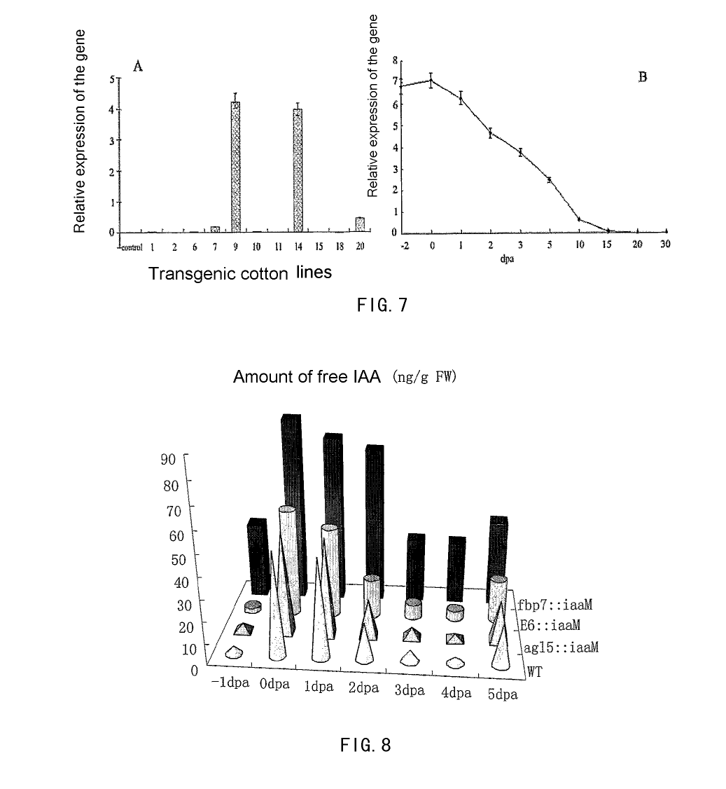

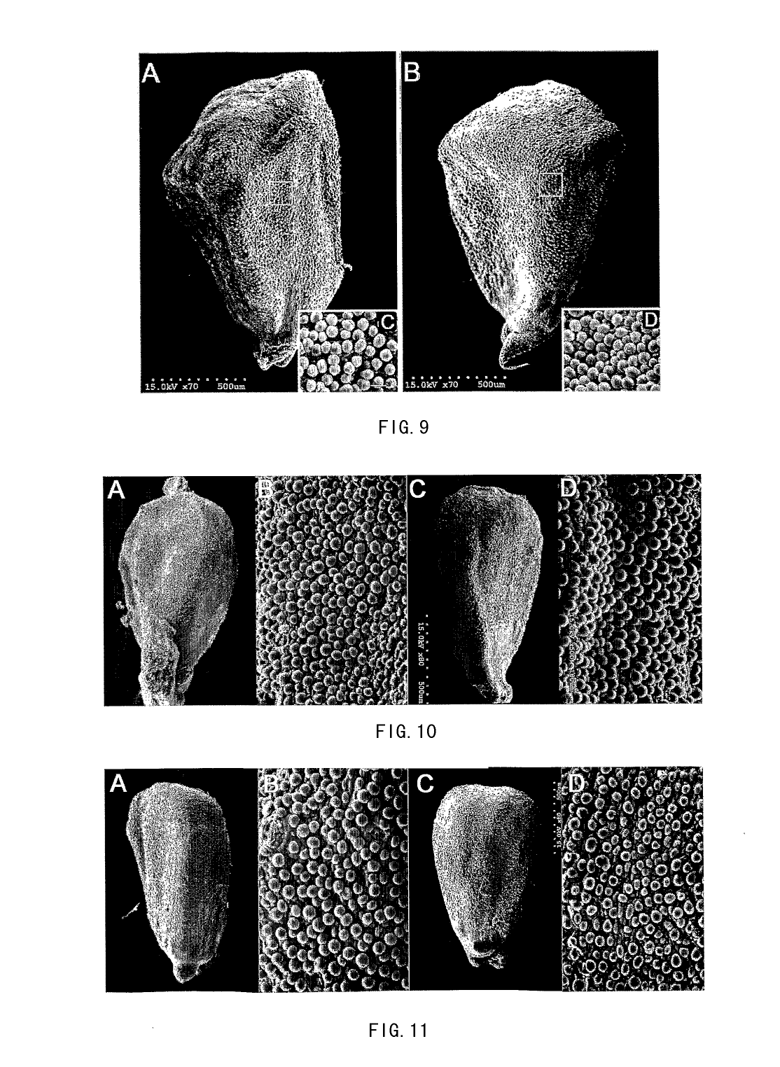

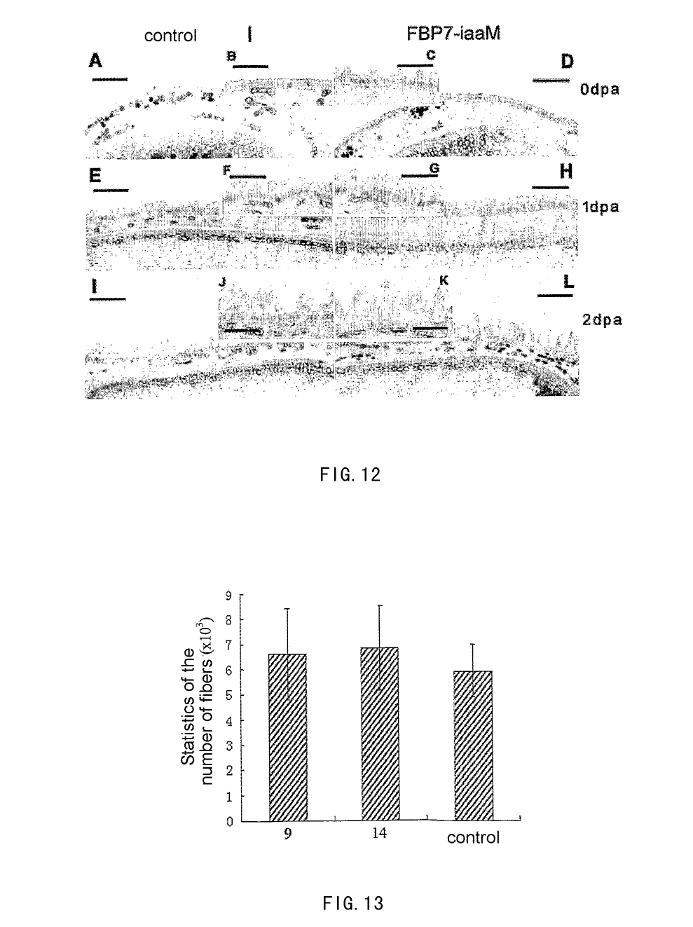

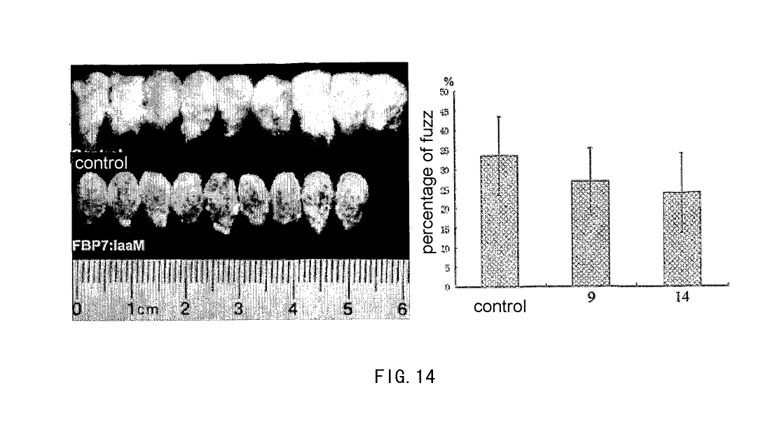

The vector constructing process is shown in FIG. 1. The initial plasmid vectors were respectively from 1 and 2 above. p5 was obtained by modification on the basis of pBI121 (Clontech, Ltd.), using the methods in Molecular Cloning: A Laboratory Manual (Sambrook and Russell, 2001). All the restriction enzymes were purchased from Roche, used in accordance with operating instructions.

The structure of the constructed plant expression vector containing the specific promoter FBP7 is shown in FIG. 2, which includes the nucleotide sequence (SEQ ID NO. 13) expressing auxin synthesis related gene and the elements required for expression screening.

### Example 3

### The Preparation of Transformants and Transgenic Plants

1. The Constructed Plant Expression Vector Plasmid was Introduced into Agrobacterium LBA4404 by the Electric Shock Method

With reference to Bio-RAD Micropulser user manual, the vector above was introduced into Agrobacterium LBA4404 through electric shock.

2. Integrating the Vector Specifically Expressing the Plant Hormone Synthesis Related Gene into the Cotton Genome

Perform the genetic transformation of cotton by Agrobacterium tumefaciens mediated method.

TABLE 1
culture medium for genetic transformation of cotton
mediated by Agrobacterium tumefaciens

| Medium Name | Components |
|---|---|
| Basic medium. | MSB (MS inorganic salts + B5 organic) |
| Medium for seed | ½ MSB + 30 g/L glucose + 7.5 g/L agarose, pH 6.5 |
| germination | |
| Callus induction | MSB + 0.5 mg/L IAA + 0.1 mg/L Kt + 30 g/L glucose + 2.0 g/L |
| medium | Gelrite, pH 5.8. |
| Embryogenic callus | MSB + 1.9 g/L KNO₃ + 30 g/L glucose + 2.0 g/L Gelrite, |
| induction medium | pH 5.8 |
| Co-culture medium | MSB + 1.9 g/L KNO₃ + 30 g/L glucose + 2.0 g/L Gelrite + 100 |
| | μmol/L acetosyringone, pH 5.2 |
| Screening medium | MSB + 1.9 g/L KNO₃ + 75 mg/L kanamycin + 500 mg/L |
| | Cefotaxime + 30 g/L glucose + 2.0 g/L Gelrite, pH 5.8 |
| Liquid suspension | MSB + 1.9 g/L KNO₃ + 30 g/L glucose, pH 5.8 |
| medium | |
| Somatic embryos | MSB + 1.9 g/L KNO₃ + 30 g/L glucose + 2.0 g/L Gelrite, pH 6.5 |
| maturation medium | |
| Somatic embryos | ½ MSB + 15 g/L glucose + 4.0 g/L Gelrite, pH 6.5 |
| elongation medium | |
| Seedling medium | SH medium + 0.05% activated carbon + 20 g/L sucrose, pH 6.5 |

MS: Murashige & Skoog, 1962

B5: Gamborg, 1986

Gelrite: Sigma, Product No.: G1910

SH: Schenk & Hildebrandt, 1972

The expression vector above was introduced into cotton by Agrobacterium mediated method of embryogenic callus. Specific steps were as follows:

(1) Inducing cotton embryogenic callus: husk cotton seeds (Hebei cotton cultivar Jimian 14), sterilize with 0.1% mercuric chloride (HgCl₂) for 10 mins, rinse 6 times with sterile water, and sow seeds in the seed germination medium (for components of the medium, see table 1). Germinate for 5-7 days at 28° C. in the dark to obtain sterile cotton seedlings. Select the hypocotyls of sterile seedlings which are healthy and strong, cut them into pieces of about 0.4 cm long, inoculate them on the cotton callus induction medium to induce callus, culture them at 28° C. for 16 h in light. Subculture once every 3-4 weeks. Select the loose callus to be inoculated onto the embryogenic callus induction medium to induce the production of embryogenic callus, and culture at 28° C. for 16 h in light.

(2) Culturing the transformed Agrobacterium: pick out the Agrobacterium strains containing the expression vector above, and carry out the streak culture on the YEB solid medium (0.5% sucrose (W/V), 0.1% yeast extract for bacteria (W/V), 1% of bacto-tryptone (WN), 0.05% MgSO₄.7H₂O (W/V), 1.5% of the agarose powder(WN) pH7.0) containing 50 mg/L kanamycin and 125 mg/L streptomycin. Pick out Agrobacterium single colonies and inoculate them into 5 mL YEB liquid medium containing the same antibiotics, culture at 28° C. with agitation at 200 r/min, overnight. Transfer the cultured Agrobacterium broth at a ratio of 1:20 to the 50 mL YEB liquid medium with the same antibiotics, and continue to culture at 28° C. with agitation at 200 r/min for 3-5 h. Centrifuge at 6,000 r/min for 10 min, and then resuspend the bacteria with liquid MSB culture medium (containing 100 μmol/L acetosyringone). Adjust the OD₆₀₀ value to 0.3-0.5 for use.

(3) Dipping and co-culturing: absorb the liquid from the surface of embryogenic callus with sterile absorbent paper, place it into the Agrobacterium broth with adjusted concentration, then dip for 20-30 mins. Dispose of the broth, transfer the dipped embryogenic callus to the co-culture medium, and co-culture in the dark at 24° C. for 4 days.

(4) screening for transformants: after co-culturing, transfer the embryogenic callus to the screening medium for removing bacteria and selectively culturing, culture at 28° C. for 16 h in light, and subculture once every 3 weeks. 1-2 months later, most of the callus browned and died, a small part showed kanamycin resistance and fresh embryogenic callus grew. Subculture the callus blocks. When each block of tissue proliferated to 2.0-3.0 g, transfer it into the liquid suspension medium, culture in suspension on the shaking bed with agitation at 120 r/min, to obtain a large number of somatic embryos. After suspension of 2 weeks, filter the suspension culture tissue with a 30-mesh sieve, the filtered sediments were transferred to the somatic embryo maturation medium. Mature embryos which germinate were transferred to the Somatic embryos elongation induction medium. Culture in the dark at 28° C. for 2 weeks to induce somatic embryo elongation and germination. Take larger germinating embryos (>0.5 cm) and transfer to the SH medium for seedling, culture at 28° C. for 16 h in light. When the seedlings grew to about 2 cm high, clip the seedlings and graft them onto a cotton seedling having 3 to 4 true leaves.

b 3. Obtaining Cotton with Improved Fiber Trait

Culturing of grafted cotton was conventionally managed in the greenhouse. After maturation, seeds and fibers were collected for traits analysis of yield and quality. The resultant transgenic cotton and the wild-type control were not significantly different in terms of phenotype and growth and development.

### Example 4

### Detecting the Expression of the Introduced Auxin Synthetase Gene iaaM in Cotton by the RT-PCR Method

A First Strand cDNA Synthesis Kit (MBI Ltd.) was used to synthesize first strand cDNAs of various RNAs, and the operations were carried out according to the instructions for the kit. 1 μL of a first strand product as template was used for PCR amplification. The 25 μL system included 1×PCR buffer, 0.2 mmol/L dNTPs, 1.5 mmol/L MgCl₂, 0.2 μmol/L of each of the upstream and downstream primers of iaaM gene (SEQ ID NOs. 14 and 15), and 1 U Taq DNA polymerase (Promega). The temperature cycle parameters were: predenaturing, 94° C., 5 mins; 94° C., 30 secs, 56° C., 30 secs, 72° C., 1 min, 30 cycles; extension, 72° C., 5 mins. Histone3 gene of histone was used as an internal standard. For the primer sequences 16 and 17 of Histone3 of histone, see Zhu Y Q et al, 2003, Plant Physiology, 133, 580-588. RT-PCR results are shown in FIGS. 4-6.

### Example 5

### Detecting the Expression of the Introduced iaaM Gene in Cotton Fiber by the Real-Time PCR Method

Extract the total RNAs from the ovules and fibers 10 days after flowering of the control and the transgenic cotton, and then synthesize first strand cDNAs by reverse transcription which were used as templates for the quantitative real-time PCR amplification. Specific steps were as follows: a First Strand DNA Synthesis Kit (MBI Fermentas) was used to synthesize first strand cDNAs of various RNAs, and the operations were carried out according to the instructions for the kit. PCR was carried out with a quantitative real-time PCR device. The 25 μL system included 12.5 μL MIX buffer (Bio-Rad, including PCR buffer, DNA polymerase, dNTPs and MgCl₂), 0.2 μmol/L of each of the upstream and downstream primers, and 1 μL of first strand product. The temperature cycle parameters were: predenaturing, 94° C., 3 mins; 94° C., 30 secs, 56° C., 30 secs, 72° C., 0.5 min with the predetermined cycle number of 35. The cotton Histone3 gene was used as the internal standard. The upstream and downstream primers were SEQ ID NOs: 16 and 17. The upstream and downstream primers for amplifying iaaM gene were SEQ ID NOs.14 and 15. Before quantitative real-time PCR, the same primers and templates were amplified once in the same temperature control program. By agarose gel electrophoresis, examine and ensure that the amplification products were single-band. The results are shown in FIG. 7.

### Example 6

### Examining the Quantitative Trait of Cotton Fiber Specifically Expressing the Plant Hormone Synthetase Gene iaaM

### 1. Observing by Scanning Electron Microscope

In accordance with the conventional sample preparation method for electron microscope (Preparation Technologies of Biological Samples for Electron microscope, Huang Li, Nanjing: Jiangsu Science and Technology Press, 1982), select the ovules on the flowering day, which were fixed by a succession of glutaraldehyde, osmic acid and tannin, and subjected to serial gradient dehydration with ethanol, replacement, drying, ion plating, and then the surface observation by scanning electron microscope. The initiation situations of fibers on the ovule surfaces of the prepared samples were observed under a HITACHI S-3000N SEM scanning electron microscope. The results were shown in FIG. 9. The distribution of the fiber primordia on the ovule surfaces of the transgenic cotton was denser than that of the wild type, and the number thereof was larger.

### 2. Early Fiber Count

Select the cotton bolls two days after flowering. Select randomly 3 individual plants for each line. Take 5 bolls from each plant, and 2 seeds per boll for counting the fibers on the surfaces of ovules. Count 10 times for each sample, and calculate the average number as the early fiber number of the individual plant. The specific steps were as follows:

Take 2 full ovules two days after flowering, place them in a 1.5 ml centrifuge tube, and add a suitable amount of FAA fixative solution for fixing for more than one hour. Rinse twice with deionized water, add 200 μL 5 mol/L HCl, and dissociate at room temperature for more than one hour. Dispose of HCl, rinse twice with deionized water, and add 100 μL Schiff reagent for treating more than one hour. Remove the Schiff reagent, rinse twice with deionized water, add 100 μL 45% acetic acid and grind with a glass rod so that fibers fell from the ovule surfaces and scattered. The ground fiber solution was mixed uniformly with a pipette, place the solution in a blood cell counting plate, and observe it under the microscope and count. The results were shown in FIG. 13. The early fiber number of the transgenic cotton was increased significantly.

### 3. The Microscopic Section Observation of Early Ovules

Select the ovules at different time after flowering for paraffin sections, in order to observe the impacts of the specific expression of the iaaM gene in ovules on the growth and development of cotton fibers at the tissue level. Take the ovaries at different time after flowering (including ovules) from the plants and immediately cut off the tops, divide them into small scraps or small pieces of about 0.5 cm, and make the sections by the conventional methods of tissue sections. The sections were observed with OLYMPUS BX41TF microscope and photographed. The results were shown in FIG. 12. The protrusions of fiber primordia could be obviously observed on the 0 dpa ovule surfaces of the transgenic cotton, while the control surfaces were relatively smooth with a small number of protrusions which were not obvious. On the 1 dpa ovule surfaces, protruding fiber cells could be clearly observed on both the control and the transgenic cotton, wherein the number of the fiber cells of the ovule surfaces of the transgenic cotton was significantly larger than that of the control. The observation results of 2 dpa ovule surfaces also showed that the number of fiber cells was remarkably increased, wherein the fibers of the transgenic cotton were significantly longer than those of the control.

### Example 7

### Examining the Amounts of IAA in Cotton Ovule and Fiber Specifically Expressing the Plant Hormone Synthetase Gene iaaM

With [¹³C₆]IAA as the internal standard, the high-pressure liquid chromatography-mass spectrometry analysis was used.

1. The preparation of the internal standard: the internal standard [¹³C₆]IAA was dissolved in the appropriate volume of 100% methanol to prepare a stock solution having a final concentration of 500 ng/μL
2. Hormone extraction and purification: Take cotton ovules/fibers, which were frozen in liquid nitrogen quickly and ground into powder. Accurately weigh about 0.5 g of sample powder (by subtraction), add 7 ml extract liquid (80% pre-cooled methanol), and 10 ng of the internal standard [¹³C₆]IAA (dissolved in 100% methanol, final concentration of 500 ng/μL). Seal the mixture in a glass tube after mixing, extract in the dark at −20° C., overnight. The extracted sample was transferred into the centrifuge tube, centrifuge at 10,000 g at 4° C. for 20 mins. Extract the supernatant to a distillation flask, and add a drop of ammonia to make the solution pH basic at 40° C. The solution was subjected to vacuum rotary evaporation for condensation in a 100 mL distillation flask until it was dried. The sample was re-dissolved with 5 mL 0.1M HAC. PH was measured again after dissolution, remaining consistent with the redissolved solution (0.1M HAC). Column chromatography purification: column chromatography with 5 mL 100% methanol, activate the extraction column (Sep-Paka Cartridges, Waters); pass 10 mL 0.1M HAC at twice through the column, wash away methanol, and balance the extraction column; load the sample, slowly pass the sample through the column; pass 4 mL 17% methanol (0.1M HAC dilution) slowly through the column for rinsing; elute the sample with 6 mL 40% methanol (0.1M HAC dilution), and collect the filtered liquid. The collected liquid was subjected to vacuum rotary evaporation for concentration in a 50 mL distillation flask at 40° C. until dried. The sample was re-dissolved with 1.5 mL 80% methanol, and then transferred to a 1.5 mL centrifuge tube, subjected to vacuum drying for concentration. The dried sample was sealed and kept at low temperature from light.
3. Hormone detecting: the detecting equipment used was high pressure liquid chromatograph-mass spectrometer produced by Shimadzu (LCMS-2010A). The dried sample was re-dissolved with 10% methanol, applied by the trace sample applicator, 10% methanol, 5 mins, 85% methanol, 30 mins, 100% methanol, 31 mins, 100% methanol, 39 mins; 10% methanol, 40 mins; 10% methanol, 60 mins.

Record the retention time of the chromatographic peak, the peak area and the mass spectrogram of the internal standard [¹³C₆]IAA. According to the retention time and the mass spectrogram, identify IAA in individual samples, and calculate the chromatographic peak areas of the internal standard IAA and the endogenous IAA. Repeat three times, and take the average value of the peak areas. Calculate the amounts of IAA in the samples by the internal standard method. The results were shown in FIG. 8.

### Example 8

### Examining the Lint Percentage and Quality of Cotton Fiber Specifically Expressing the Plant Hormone Synthetase Gene iaaM

1. The Comparative Analysis of the Amount of the Lint Percentage and Fiber Yield of the Transgenic Cotton with the Control

The transgenic cotton plants of T₁ generations were grown in the transgene base of the Southwest University with the random plot design. Three plots were set up for each line with the conventional field management. Harvest the mature cotton by plot, and accurately weigh the seed cotton yields. Randomly select 100 seeds from the seed cotton harvested from each plot, then accurately weigh the weights of the 100 seed cotton. Take off fibers by hand and then weigh the total amount of fibers and the total amount of seeds respectively to calculate the lint percentage. Finally, take the average values of three plots as the final results (Table 2). According to the experimental results it can be seen that: the seed cotton yield and the lint percentage of the plot of the non-transgenic control were 3.37 Kg and 42.19% respectively, while the lint percentages of FBP7-iaaM transgenic lines were all above 43%, up to 49.67%, which were significantly greater than that of the control; and the lint percentages of E6-iaaM and AGL5-iaaM transgenic plants were lower. The lint cotton yield of FBP7-iaaM transgenic lines was higher than that of the control, indicating that FBP7-iaaM transgenosis could increase the cotton fiber yield. The lint cotton yields of E6-iaaM and AGL5-iaaM transgenic plants were lower than that of the control. The weights per 100 seeds of all the transgenic lines were not significantly different from those of the control.

TABLE 2
The comparison of the fiber yields of
the transgenic cotton with the control

| | | | Seed cotton | Lint cotton |
|---|---|---|---|---|
| | Weight per | Lint per- | yield | yield |
| Gene Name | 100 seeds (g) | centage % | (Kg/Plot) | (Kg/Plot) |
| Control | 10.34 | 42.19 | 3.37 | 1.42 |
| FBP7 - iaaM - 9 | 10.44 | 48.62 | 3.39 | 1.65 |
| FBP7 - iaaM - 14 | 10.30 | 49.67 | 3.14 | 1.56 |
| FBP7 - iaaM - 20 | 10.84 | 43.91 | 3.47 | 1.52 |
| AGL5 - iaaM - 6 | 10.92 | 41.53 | 2..74 | 1.14 |
| AGL5 - iaaM - 10 | 11.07 | 42.82 | 2..84 | 1.22 |
| E6 - iaaM - 4 | 10.31 | 37.70 | 2..81 | 1.06 |
| E6 - iaaM - 7 | 10.23 | 41.70 | 2..78 | 1.16 |
| E6 - iaaM - 19 | 10.52 | 37.50 | 2.84 | 1.07 |

Control: the GUS staining negative plants isolated from the progeny generation as the control.

2. The Reduction of Seed Fuzz of Fbp7-iaaM Transgenic Cotton

The cotton seeds were delinted by a delinter, and then stay under the environmental condition of 65% relative humidity at 20° C. for more than two days. After weighing the delinted cotton seeds, the seeds were delinted with concentrated sulfuric acid, then weighed again after drying and staying under the environmental condition of 65% relative humidity at 25° C. for more than 24 hrs. The calculated weight difference was the fuzz weight. The fuzz content was represented by percentage. The results were shown in FIG. 14. By comparing the seed weights before and after delinting, it was found that for the control the fuzz weight accounted for about 34% of the total weight of the seeds, while for transgene 9^{#} and 14^{#}, the weight was 24%-27%, significantly less than that of control.

### 3. The Quality Test of the Transgenic Cotton Fiber

The cotton samples were sent to the Testing Center for Quality Supervision and Detection of Cotton Quality of Ministry of Agriculture (Anyang) for testing under the environmental condition of 65% relative humidity at 20° C. by HFT9000 in accordance with ASTM D5867-95 “Test Methods for HVI900 High Volume Testing Instruments for Fiber”, in terms of five indices, namely length, uniformity, specific breaking strength, elongation and micronaire value. The GUS negative plants isolated from the T₁ generation were used as the control, which did not contain the transgenic components. The results were shown in Table 3.

TABLE 3
The comparison of the fiber qualities of
the transgenic cotton with the control

| | | | Specific | | |
|---|---|---|---|---|---|
| | | Unifor- | breaking | Elonga- | |
| | Length | mity | strength | tion | |
| Gene Name | (mm) | (%) | (cN/tex) | (%) | Micronaire |
| Control | 30.41 | 85.16 | 30.08 | 5.76 | 5.25 |
| FBP7 - iaaM - 9 | 30.21 | 86 13 | 29.97 | 6.09 | 4.62 |
| FBP7 - iaaM - 14 | 29.92 | 85.89 | 30.74 | 5.88 | 4.51 |
| FBP7 - iaaM - 20 | 30.39 | 86.39 | 29.99 | 5.98 | 5.16 |
| AGL5 - iaaM - 6 | 30.5 | 86.95 | 31.32 | 5.75 | 5.03 |
| AGL5 - iaaM - 10 | 30.31 | 86.45 | 30.38 | 5.95 | 5.11 |
| E6 - iaaM - 4 | 30.13 | 85.45 | 30.75 | 6.00 | 5.17 |
| E6 - iaaM - 7 | 29.83 | 85.70 | 29.50 | 6.25 | 5 28 |
| E6 - iaaM - 19 | 29.86 | 85.10 | 30.07 | 6.00 | 5.08 |

The length of the transgenic cotton fibers was not significantly different from that of the control. The uniformity of the transgenic fibers was 85.70%-86.95%. T-test results showed no significant difference from the control. The results of the two indicators related to fiber strength, namely specific breaking strength and elongation, both showed that the fiber strengths of the transgenic lines and the control were not notably different. The test results of the micronaire value relevant to fiber fineness and maturity showed that the micronaire value of FBP7-iaaM transgenic cotton was significantly reduced as compared with the control. According to the National Classification Standard of Cotton, in light of micronaire value differences, cotton is classified into three classes, namely A, B, C, with B for the standard class. The numerical range of A is 3.7-4.2, representing the best quality; the numerical range of B class is 3.5-3.6 and 4.3-4.9; the numerical range of the C class is 3.4 and below and 5.0 and above, representing the worst quality. The micronaire values of the transgenic cotton fibers were in the B1 and the B2 ranges, belonging to the standard class. The micronaire values of the control and the E6-iaaM and AGL5-iaaM transgenic cotton fibers were significantly higher, falling into the C class and having poor quality. The deviation of the fiber micronaire value of the control from the normal range was associated with the climate conditions in the cultivation year (2007) of the trial area for the field tests (Chongqing). The weather conditions of high temperature and little rain, long time sunshine, intensive sunlight, and low temperature difference between day and night affect the maturity and fineness of cotton fiber. However, under the same cultivation conditions, FBP7-iaaM transgenic plants still had the micronaire value regarding fiber fineness and maturity obviously superior to that of the control, indicating that the transgenic plants have the improved quality of fibers. However, the E6-iaaM and AGL5-iaaM transgenic plants, as compared with the non-transgenic control, did not have the remarkably different micronaire values.

In conclusion, with reference to the test results of the fiber quality, FBP7-iaaM transgenic cotton was not significantly different from the control in terms of fiber length and strength, but the fiber fineness of it is less than that of the control, demonstrating the improvement of its quality.

### INDUSTRIAL APPLICABILITY

The examples above show that the method of improving cotton fiber trait in the present invention can achieve the specific expression of auxin synthesis related gene at the specific parts of cotton at the particular developmental stages, and thus achieve the endogenous modulation of the auxin amount in the particular tissues and organs of cotton, thereby fulfill the purposes of improving cotton fiber yield and quality (fineness and strength). The experimental results demonstrate that for the cotton modified by the method in this invention of improving cotton fiber trait, the number of bolls is increased, the seed number is raised, the number of cotton fibers is obviously raised, the lint percentage is significantly increased, the fiber yield is dramatically increased, while the cotton fiber strength remains unchanged and fineness and maturity are improved. The method of the invention can be easily carried out with prominent effects, offering a promising market.

1. A plant expression vector expressing auxin synthesis related gene, at least comprising a nucleotide sequence expressing auxin synthesis related gene and consisting of a plant auxin synthetase gene and a plant seed coat-specific promoter.

2. A plant expression vector according to claim 1, characterized in that
the nucleotide sequence at least comprises a plant auxin synthetase gene iaaM and a plant seed coat-specific promoter which is FBP7 gene promoter, wherein the FBP7 gene promoter is located upstream of the 5′ end of the plant auxin synthetase gene iaaM.

3. A plant expression vector according to claim 1, characterized in that the nucleotide sequence has the sequence as represented by SEQ ID NO: 13.

4. A plant expression vector according to claim 1, characterized in that the plant expression vector has the structure as shown in FIG. 2.

5. A transformant obtained by transforming a host with the plant expression vector according to claim 1.

6. Use of the plant expression vector according to any one of claims 1-4 in improving cotton fiber trait.

7. A method of producing a transgenic plant comprising the plant expression vector according to claim 1, comprising the following steps:
1) operably linking the plant auxin synthetase gene with the plant seed coat-specific promoter;
2) constructing the plant expression vector comprising the plant auxin synthetase gene and the plant seed coat-specific promoter;
3) transforming a host by using the plant expression vector to obtain the transformant;
4) transforming a plant by using the transformant to obtain the transgenic plant.

8. A nucleotide sequence encoding and expressing auxin synthesis related gene, characterized in that it has the nucleotide sequence as represented by SEQ ID NO: 13.
